(19) United States
(12) Patent Application Publication
(10) Pub. No.: US 2010/0119512 A1
(43) Pub. Date: May 13, 2010

(54) METHODS OF DIAGNOSING, TREATING, AND PREVENTING INCREASED VASCULAR PERMEABILITY

(75) Inventors: Edward P. Feener, North Reading, MA (US); Lloyd P. Aiello, Belmont, MA (US)

Correspondence Address: CLARK & ELBING LLP, 101 FEDERAL STREET, BOSTON, MA, 02110, US

(73) Assignee: Joslin Diabetes Center, Boston, MA (US)

(21) Appl. No.: 12/522,625

(22) PCT Filed: Jan. 25, 2008
(86) PCT No.: PCT/US08/00998

§ 371 Date: Jan. 8, 2010

Related U.S. Application Data
(60) Provisional application No. 60/897,387, filed on Jan. 25, 2007.

(51) Int. Cl. A61K 39/395; A61K 38/00; A61K 31/155; A61K 31/662; A61K 38/07; A61K 31/415; A61K 31/42; A61K 31/35; A61K 31/433; A61P 27/02

(52) U.S. Cl. 424/135.1; 514/2; 424/158.1; 514/637; 514/143; 514/18; 424/141.1; 424/146.1; 424/133.1; 424/136.1; 424/94.64; 514/406; 514/378; 514/455; 514/363

(57) ABSTRACT

The present invention provides methods for the treatment and diagnosis of disorders associated with excessive vascular permeability and edema.

### FIELD OF THE INVENTION

This invention relates to methods of treating and preventing increased vascular permeability and edema, particularly to treating and preventing increased vascular permeability in the brain and retina.

### BACKGROUND OF THE INVENTION

The control of vascular permeability is essential for maintenance of normovolemia, most importantly in constrained spaces of the body such as the eye and the brain. Vasogenic cerebral edema arises from transvascular leakage caused by mechanical failure or dysfunction of the endothelial tight junctions of the blood-brain barrier (BBB), and is characterized by an increase in extracellular fluid volume due to the increased permeability of brain capillary endothelial cells to macromolecular serum proteins (e.g., albumin). Under normal physiological conditions, the entry of plasma protein-containing fluid into the extracellular space is limited by endothelial cell tight junctions. However, in the presence of massive injury there is increased permeability of brain capillary endothelial cells. Vasogenic edema can displace the brain hemisphere; severe edema can lead to cerebral herniation and contribute to neuronal cell death. Vasogenic edema is often associated with subdural hemorrhage (e.g., from a cranial injury) and hemorrhagic stroke.

Diabetic retinopathy (DR) is the leading cause of vision loss in working adults. Although its incidence and progression can be reduced by intensive glycemic and blood pressure control, nearly all patients with type 1 diabetes mellitus (DM) and over 60% of those with type 2 DM develop retinal microvascular abnormalities termed nonproliferative diabetic retinopathy (NPDR), and 20% to 30% of these patients advance to active proliferative diabetic retinopathy (PDR) and/or diabetic macular edema (DME). While photocoagulation surgery and vitrectomy are highly effective in reducing vision loss, preventative treatments for PDR and DME remain a major unmet clinical need.

Increased retinal vascular permeability (RVP) is a primary cause of DME and a characteristic finding in PDR, as well as other disorders. The retinal vascular barrier has an essential role in maintaining the composition of both of retinal interstitial fluid and the vitreous humor. An increase in RVP occurs in early diabetes and the magnitude of RVP correlates with the severity of DR. Although the etiology of DME is not fully understood, a primary cause of macular thickening appears to involve the diffusion of proteins and lipids across the retinal endothelium into the retina resulting in fluid retention and lipid exudates within the macula. Over the past decade, a number of groups have demonstrated that growth factors and hormones, including vascular endothelial growth factor (VEGF), angiotensin II, and interleukin-6, are elevated in the vitreous of individuals with PDR and DME. The vitreous also contains anti-angiogenic and anti-permeability factors, such as pigment epithelium-derived factor (PEDF) and angiostatin, which can oppose the effects of VEGF. These reports support the general proposition that vitreous fluid contains proteins that correlate with specific retinal pathologies, and that proteins in the vitreous compartment affect retinal vascular functions. A variety of retinal vascular conditions are believed to be associated with increased permeability; many of these conditions, e.g., the ischemic retinopathies, are thought to be mediated by these and other as yet unknown factors.

### SUMMARY OF THE INVENTION

The present invention is based, at least in part, on the discovery that the kallikrein/kinin pathway plays a role in vascular leakage, e.g., in the brain and retina, and that this pathway can be modulated to affect vascular permeability.

As described herein, one of the ways that this pathway can be modulated is using pH. Increased (more alkaline) pH is associated with increased permeability. Thus, the methods described herein can include interventions that reduce pH, e.g., the administration of compounds that decrease pH. In general, the methods include returning pH to approximately 7.4.

pH can also be used to evaluate a subject, e.g., to diagnose a condition associated with increased vascular permeability, or to predict a subject's risk of developing such a condition. Thus, the methods described herein can include determining the pH of a relevant fluid, e.g., the vitreous in the eye, or the CSF in the brain. In some embodiments, the presence of significantly increased pH, e.g., a pH of about 7.8 or above, is indicative of the presence of, or an increased risk of developing, a condition associated with increased vascular permeability. The pH effect on kallikrein activation may not have a specific threshold; it is likely that that the effect of increasing pH above 7.4 (e.g., any increase above 7.4) is continuous. As analogy, the effect of alkaline pH on kallikrein may be more similar to a rheostat than an on/off switch. Thus, in some embodiments, the presence of increased pH (e.g., pH above 7.5) is indicative of the presence of, or an increased risk of developing, a condition associated with increased vascular permeability.

In addition, the presence of increased pH can be used as a basis for selecting a subject, e.g., for the administration of a treatment, or for inclusion in a clinical trial. pH can also be monitored over time, e.g., to evaluate the efficacy of a treatment; an effective treatment is one that reduces pH, i.e., returns the pH to normal or substantially normal (“substantially normal,” as used herein, means not significantly different from normal, i.e., a pH of about 7.3 to 7.4).

In part, the results described herein demonstrate that the kallikrein/kinin pathway is present and active in the vitreous of human patients with proliferative diabetic retinopathy (PDR). The molecular players in the kallikrein/kinin pathway can be targeted to reduce vascular permeability. For example, prekallikrein, kallikrein, Factor XII, and high molecular weight kininogen were all found to be active in the vitreous of PDR patients (see FIG. 6). Thus, each of these proteins is a target for therapeutic intervention in the methods described herein.

Finally, the data presented herein indicates that conditions associated with increased retinal vascular permeability (RVP) may often be a result of retinal hemorrhage. Presently, retinal hemorrhages are considered to be relatively benign, and the primary course of treatment is to follow them for more severe problems. The present data suggest that events activate the kallikrein/kinin pathway and lead to increased RVP. Thus, in subjects with a history of retinal hemorrhage, the present methods include administering (either systemically or locally, e.g., to the eye, e.g., by intraocular injection), a treatment that reduces retinal hemorrhage and/or a treatment that ameliorates the effects of a retinal hemorrhage.

In one aspect, the present invention includes methods for treating or preventing the development or progression of a condition associated with increased vascular permeability in the eye of a subject. The methods can optionally include selecting a subject on the basis of one or more of the following: that they have a history of ocular hemorrhage or a condition associated with increased vascular permeability in the eye of a subject.

In some embodiments, the methods include administering to the subject a therapeutically effective amount of an inhibitor of Factor XII (FXII). Suitable inhibitors include, but are not limited to, C1 inhibitor, Corn Hageman Factor Inhibitor (CHFI), H-D-Pro-Phe-Arg-chloromethylketone (PCK), haemaphysilin, hamandrin, alpha 2-antiplasmin, alpha 2-macroglobulin, antithrombin III, ecotin XII-18, and plasma kallikrein-specific kunitz domain inhibitor (KALI-DY).

In some embodiments, the methods include administering to the subject a therapeutically effective amount of an inhibitor of prolylcarboxypeptidase, prekallikrein (PK), or high molecular weight kininogen (HK), e.g., one or more of HKH20, an inhibitory anti-PK antibody, an inhibitory anti-HK antibody, a benzamidine, a corn trypsin inhibitor, a diisopropylfluorophosphonate, a leupeptin, an anti-PRCP antibody, or a soybean trypsin inhibitor.

In some embodiments, the methods include administering one or more of a compound that inhibits binding between prekallikrein and heat shock protein 90, e.g., an antibody or antigen-binding portion thereof with binding specificity for prekallikrein or heat shock protein 90.

In some embodiments, the methods include administering to the subject a therapeutically effective amount of a compound that substantially normalizes pH in the eye of the subject, e.g., a compound selected from the group consisting of a weak acid, a buffer capable of returning the pH to the desired level, a carbonic anhydrase inhibitor, and a bicarbonate transporter inhibitor (e.g., acetazolamide, celecoxib, valdecoxib, topiramate, or zonisamide).

In a second aspect, the present invention includes methods for treating subjects who have a history of ocular hemorrhage (i.e., have had one or more hemorrhages). The methods include selecting a subject on the basis that they have had at least one ocular hemorrhage, and administering to that subject a treatment to reduce the risk of future hemorrhages. In some embodiments, the subject has an underlying medical condition selected from the group consisting of diabetes, sickle cell anemia, hypertension, or trauma. In some embodiments, the treatment is selected from the group consisting of administration of one or more of an anti-hypertensive drug, administration of a composition comprising activated Factor VII (e.g., eptacog alfa), reduction or reversal of any anticoagulation medicaments used by the patient, and administration of isotonic fluids.

In a third aspect, the present invention provides methods for determining whether a subject has or is at risk of developing a condition associated with increased retinal vascular permeability. The methods include determining the pH in the eye of the subject, e.g., in the vitreous. The presence of a pH that is significantly higher than normal indicates that the subject has or is at risk of developing a condition associated with increased retinal vascular permeability. In some embodiments, a pH above about 7.5 indicates that the subject has or is at risk of developing a condition associated with increased retinal vascular permeability. In some embodiments, a pH of about 7.8 or higher indicates that the subject has or is at risk of developing a condition associated with increased retinal vascular permeability.

The methods described herein can include administering a composition described herein by local administration to the eye of the subject, e.g., by injection into the vitreous or aqueous humor of the eye, or by intrabulbar injection, or by administration as eye drops. In some embodiments, the methods include the use of a local drug delivery device (e.g., a pump or a biocompatible matrix) to deliver the composition. In other embodiments, the composition is delivered via injection into the cerebral fluid or cerebral spinal fluid. In some embodiments, the administration is systemic.

As used herein, disorders associated with excessive vascular permeability include, but are not limited to, disorders associated with increased retinal or cerebral vascular permeability and/or vasogenic edema. Described herein are methods of treating such disorders, e.g., by decreasing vascular permeability, e.g., decreasing retinal vascular permeability in the eye of a subject or decreasing cerebral vascular permeability in the brain of a subject. In some embodiments, the methods described include a step of selecting a subject on the basis that the subject has, or is at risk for developing, a disorder associated with excessive vascular permeability, as described herein.

Disorders associated with excessive vascular permeability and/or edema in the brain include, but are not limited to, cerebral edema, intracerebral hemorrhage, subdural hemorrhage, and hemorrhagic stroke. Cerebral edema is an increase in brain volume caused by an absolute increase in cerebral tissue fluid content; vasogenic cerebral edema arises from transvascular leakage caused by mechanical failure of the endothelial tight junctions of the blood-brain barrier (BBB).

Disorders associated with excessive vascular permeability and/or edema in the eye, e.g., in the retina or vitreous, include, but are not limited to, age-related macular degeneration (AMD), retinal edema, retinal hemorrhage, vitreous hemorrhage, macular edema (ME), diabetic macular edema (DME), proliferative diabetic retinopathy (PDR) and nonproliferative diabetic retinopathy (DR), radiation retinopathy, telangiectasis, central serous retinopathy, and retinal vein occlusions. Retinal edema is the accumulation of fluid in the intraretinal space. DME is the result of retinal microvascular changes that occur in patients with diabetes. This compromise of the blood-retinal barrier leads to the leakage of plasma constituents into the surrounding retina, resulting in retinal edema. Other disorders of the retina include retinal vein occlusions (e.g., branch or central vein occlusions), radiation retinopathy, sickle cell retinopathy, retinopathy of prematurity, Von Hipple Lindau disease, posterior uveitis, chronic retinal detachment, Irvine Gass Syndrome, Eals disease, retinitis, and/or choroiditis.

Other disorders associated with increased permeability include, but are not limited to, excessive vascular permeability associated with hypertension or inflammation; increased systemic vascular permeability, e.g., associated with septic shock, scurvy, anaphylaxis, and hereditary or acquired angioedema (both of which have been linked to C1 inhibitor deficiency). In some embodiments, the disorders associated with vascular permeability that are treated by a method described herein exclude hereditary or acquired angioedema.

In some embodiments, the disorder associated with increased permeability is also associated with hemorrhage, i.e., bleeding into the affected area. In some embodiments, the disorder associated with increased permeability is also associated with lysis of erythrocytes in the affected area.

In some embodiments, the disorder associated with increased permeability is also associated with an increased volume of fluid in the tissue, e.g., edema, and the methods described herein result in a reduction in the volume of fluid. Generally, the fluid is extracellular. Thus, included herein are methods for reducing the fluid volume in a tissue.

Also provided herein are methods for identifying candidate compounds for the treatment of a disorder associated with excessive vascular permeability. The methods include providing a model of a disorder associated with excessive vascular permeability, e.g., a model of diabetic retinopathy/retinal vascular permeability or of hemorrhagic stroke; contacting the model with a test compound; detecting a level of activation of one or more of prekallikrein, kallikrein, Factor XII, or HMW kininogen; and comparing the level of activation to a reference. A test compound that causes a significant difference in activity as compared to the reference, e.g., a decrease, is a candidate compound for the treatment of a disorder associated with excessive vascular permeability.

By “antibody or antigen-binding portion thereof” is meant any monoclonal antibody, polyclonal antibody, humanized antibody, a chimeric antibody, a single-chain Fv molecule, a bispecific single chain Fv ((scFv′)₂) molecule, a diabody, a triabody, a Fab fragment, a F(ab′)₂ molecule, or tandem scFv (taFv) fragment with, for example, binding specificity for prekallikrein or heat shock protein 90.

By “normal pH” is meant a pH of about 7.4, e.g., a value not significantly different from 7.4. The determination of a threshold level for elevation can be performed using standard statistical methods. In some embodiments, the presence of a pH of about 7.8 or above is considered significantly elevated.

“Substantially normal pH” as used herein, means not significantly different from normal, i.e., about pH 7.3 to 7.4. In general, a “normal” pH is about 7.4, e.g., is not statistically significantly different from 7.4.

By “vitreous hemorrhage” is meant the presence of extravasated blood within the space defined by the zonular fibers and posterior lens capsule anteriorly, the nonpigmented epithelium of the ciliary body laterally, and the internal limiting membrane of the retina (lamina limitans interna) posteriorly and posterolaterally. Distinguishing blood between the internal limiting membrane and the retina's nerve fiber layer (a subinternal limiting membrane hemorrhage) from retrohyaloid (subhyaloid) hemorrhage is not always possible, thus both conditions are generally considered to be types of vitreous hemorrhage.

By “retinal hemorrhage” is meant the presence of extravasated blood in the retina, and can be associated with trauma or an underlying medical condition, as above. Ischemic retinal vein occlusion (hemorrhagic retinopathy) is one type of retinal hemorrhage, with very poor prognosis.

By “subject” is meant either a human or non-human animal (e.g., a mammal).

Methods and materials are described herein for use in the present invention; other suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 is a bar graph illustrating the effect of intravitreal injection of anti-prekallikrein antibody (5 μl, 0.66 mg/ml) and normal mouse IgG (5 μl, 0.66 mg/ml) on CA-I- and VEGF-stimulated RVP. RVP was quantified using vitreous fluorophotometry in panels a-d & f-h. Data represent means±s.d. * P<0.05, ** P<0.01, *** P<0.001 vs. BSS, and ## P<0.01 vs. or IgG⁺ CA-I.

FIG. 2 is a line graph illustrating the effect of varying concentrations of C1-INH on PK activation by FXII in the presence of HK. Kallikrein activity was measured as cleavage of synthetic fluorogenic kallikrein substrate (0.4 mM) on a microplate reader at 37° C. at 15 minutes (means±s.e.m.; n=4).

FIG. 3 is line graph illustrating the effect of varying concentrations of anti-PK antibody on PK activation by FXII in the presence of HK (means±s.e.m. n=5-6).

FIG. 4 is a bar graph illustrating the effect of CA-I on vitreous pH measured by micro-electrode (dark bars) and fluorescent indicator (BCECF; light grey bars). * P<0.05 vs. BSS. Data represent means±s.d.

FIG. 5 is a set of four photomicrographs illustrating the effect of intravitreal injections of BSS pH 7.4 and BSS pH 8.0 on RVP. RVP was visualized using fluorescein angiography. Representative images from at least n=3 are shown. Scale bar=200 μm.

FIG. 6 is a Western blot illustrating the results of analysis of PK/kallikrein, FXII/FXIIa, and HK heavy chain in vitreous (4 μL, 1 μL, and 0.25 μL, respectively) from 6 patients with PDR. Purified standards of PK or FXII (20 ng) alone and the reaction mixture generated from a 1 hour, room temperature incubation of PK, FXII, and HK to generate kallikrein and FXIIa are shown in right lanes.

FIG. 7 is a line graph illustrating the effect of pH on PK activation in the presence of FXII and HK.

FIG. 8 is a line graph illustrating the effect of pH on FXII activation in the presence of PK and HK.

FIG. 9 is a line graph illustrating the effect of pH on kallikrein activity (solid lines) and PK autoactivation (dashed lines) in the absence of FXII and HK.

FIG. 10 is a bar graph illustrating the effect of pH on FXIIa formation from FXII by kallikrein in the presence of HK and kaolin. A Western blot of FXIIa and bar graph quantitation of FXIIa generation is shown above the bar graph. *P<0.05 vs. pH 7.4.

FIG. 11 is a line graph illustrating the effect of pH on FXII activation by kallikrein in the presence of HK and kaolin. Data represent means±s.e.m. of at least three independent experiments.

FIG. 12 is a bar graph illustrating the effect of pH, CA-I and HCO3- on PK activation by FXII in the presence of HK (means±s.e.m.; n=3-8).

FIG. 13 is a bar graph illustrating the effect of pH on PK activation by FXII in the presence of HK. ** P<0.01 vs. pH 7.4 (means±s.e.m.; n=3).

FIG. 14 is a line graph illustrating the effect of pH on FXII autoactivation in the absence of PK and HK (means±s.e.m.; n=3).

FIG. 15 is a model of carbonic anhydrase-induced permeability. Dashed arrows indicate the possible presence of one or more unknown intermediaries; solid arrows represent what is believed to be a direct connection.

### DETAILED DESCRIPTION OF THE INVENTION

The present inventors have discovered that the kallikrein pathway is present and active in the vitreous of patients with proliferative diabetic retinopathy (PDR), and that this pathway plays a role in carbonic anhydride I (CA-I)-induced vascular permeability. Furthermore, the activity of this pathway can be modulated by specific inhibitors and by pH, thereby reducing vascular permeability. Specifically, the results presented herein demonstrate that the pathway can be targeted at the level of prekallikrein (PK), e.g., using an anti-PK antibody or C1-INH, or by inhibiting the activity of Factor XII (FXII), a PK-activating protease, or by antagonism of bradykinin receptor 1 and bradykinin receptor 2.

Although the methods of the present invention are applicable to any confined anatomical space in which an increase of fluid results in increased pressures and edema, the eye is a prime example of a tissue that can be treated with the methods described herein. The methods can be used, for example, to treat a subject who has had an ocular hemorrhage, e.g., a retinal or vitreous hemorrhage.

Presently, standard treatment of an ocular hemorrhage varies depends upon the underlying cause of the bleed. In cases where there is a physical cause, such as retinal tears or detachment, surgical intervention such as laser, cryotherapy or scleral buckle surgery is often used. In the presence of underlying, non-surgical medical diseases, such as diabetes, peripheral neovascularization, or sickle cell disease, patients are generally treated conservatively as outpatients, instructed, for example, to sleep in an upright position to enhance resolution of the hemorrhage. However, in any case where the vitreous hemorrhage does not clear, pars plana vitrectomy surgery can be performed. Corticosteroids can also be prescribed.

### Increased Vascular Permeability as a Sequela of Hemorrhage

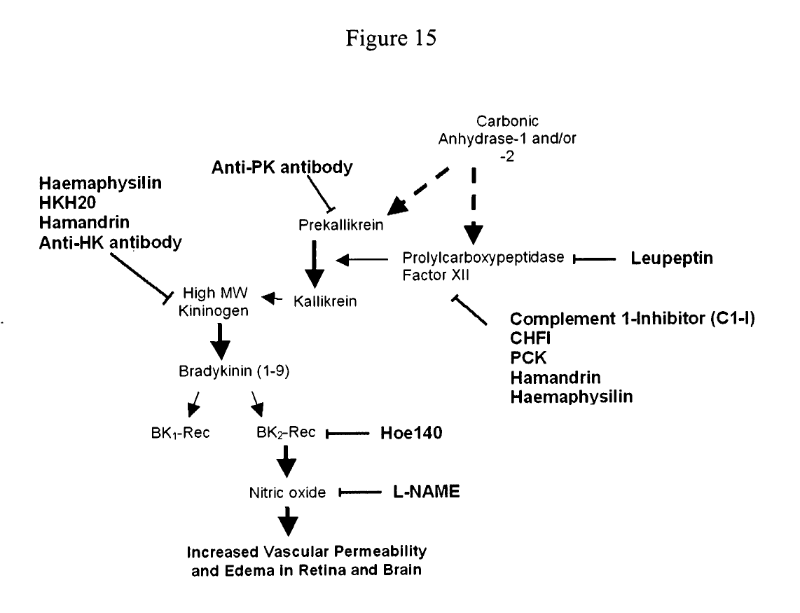

Contact activation is dependent upon the interaction of FXII, PK, and HK; as described herein and in International Patent Application Serial No. PCT/US2006/005395, activation can be initiated by increased levels of CA-I. Although not wishing to bound by theory, CA-I released by lysed red blood cells in the aftermath of a hemorrhage, e.g., in the eye or brain, can lead to activation of kallikrein signaling and subsequently to increased vascular permeability. See FIG. 15, which represents a model of this system.

In light of the data described herein, the present methods indicate administration of a treatment to reduce kallikrein signaling, thereby reducing any CA-I induced increases in vascular permeability. In the context of the eye, this includes the local or systemic administration of an inhibitor of kallikrein signaling, e.g., an inhibitor of FXII or PK as described herein. The methods can include selecting a subject on the basis that they have a history of vitreous hemorrhage, e.g., have had at least one vitreous hemorrhage.

### Inhibitors of FXII

As demonstrated herein, inhibitors of FXII can be used to reduce vascular permeability, e.g., in systems where vascular permeability is increased as a result of increased CA-I activity. FXII enhances kallikrein signaling by converting prekallikrein to kallikrein.

A number of inhibitors of FXII are known in the art. For example, C1-INH binds covalently to the active site of FXII. Others include the Corn Hageman Factor Inhibitor (CHFI; Behnke et al., Biochem. 37:15277-15288 (1998)), H-D-Pro-Phe-Arg-chloromethylketone (PCK; available from Bachem, Feinchemikalien AG, Switzerland; see also Bode et al., Protein Sci. 1(4):426-71 (1992) and Kleinshmitz et al., J. Exp. Med. 203(3):513-518 (2006)), haemaphysilin (Kato et al., Thromb. Haemost. 93(2):359-367 (2005)), and hamandrin (Isawa et al., J. Biol. Chem. 277(31):27651-27658 (2002)).

Other inhibitors useful in the methods described herein include alpha 2-antiplasmin, alpha 2-macroglobulin, antithrombin III (Pixley et al., J. Biol. Chem. 260:1723-9 1985 (1985)), ecotin XII-18 (Stoop and Craik, Nat. Biotechnol. 21(9):1063-1068 (2003)), and KALI-DY (Dennis et al., J. Biol. Chem. 270:25411-7 (1995)).

Additional inhibitors can be identified using assays known in the art, e.g., amidolytic assays.

### Inhibitors of Prekallikerin (PK)/High Molecular Weight Kininogen (HK)

Alternatively, the methods described herein can include the administration of an inhibitor of PK or HK. For example, HKH20, a peptide derived from HK (Nakazawa et al., Int. Immunopharm. 2:1875-1885 (2002)) can be used. Inhibitory antibodies that decrease the activity of PK or HK can also be used, see, e.g., Song et al., Blood 104(7):2065-2071 (2004), and the examples below, as can the inhibitors benzamidine and soybean trypsin inhibitor (Tans et al., J. Biol. Chem. 262(23):11308-11314 (1987)). Other inhibitors include DX-88 (Storini et al., J. Pharmacol. Exp. Ther. 318:849-54 (2006)), recombinant or purified complement 1 inhibitor (van Doorn et al., J. Allergy Clin. Immunol. 116:876-83 (2005)), ecotin-Pkal (Stoop and Craik, (2003); supra), and aprotinin (Scott et al., Blood 69:1431-6 (1987)).

FXII-independent mechanisms of PK activation can also be inhibited, including by the inhibition of prolylcarboxypeptidase (Shariat-Madar et al., J. Biol. Chem. 277:17962-17969 (2002) and Shariat-Madar et al., Blood 103:4554-4561 (2004)) using corn trypsin inhibitor, diisopropylfluorophosphonate, leupeptin, or anti-PRCP antibody (Shariat-Madar et al., (2004); supra). The interaction between prekallikrein and heat shock protein 90 can also be targeted (Joseph et al., PNAS 99:896-900 (2002)).

Additional inhibitors can be identified using assays known in the art, e.g., amidolytic assays.

### Prevention of Recurrent Hemorrhage

In some embodiments, the methods of the present invention include the selection of a subject who has had at least one ocular hemorrhage, and administering to that subject a treatment to reduce the risk of future hemorrhages. In some embodiments, the subject has an underlying medical cause associated with ocular hemorrhage, e.g., diabetes, sickle cell anemia, hypertension, or trauma.

The methods can include administering, e.g., either systemically or locally (i.e., to the eye), a treatment that reduces the risk of future ocular hemorrhage. Such treatments include those used for reducing the risk of hemorrhage in other tissues, e.g., the brain. For example, the treatment can include administration of one or more of an anti-hypertensive drug, administration of a composition comprising activated Factor VII (e.g., eptacog alfa), reduction or reversal of any anticoagulation medicaments used by the patient, and administration of isotonic fluids.

pH-Based Therapeutics

As demonstrated herein, the level of activation of the contact system is pH-dependent. Thus, methods that include acidifying the environment, e.g., in the eye, can also be used to reduce increased vascular permeability, thereby treating, or reducing the risk of developing, a disorder associated with increased RVP as described herein.

A number of methods are known in the art for modulating the pH of a fluid. In the present methods, it is desirable to acidify an alkaline fluid, e.g., the vitreous, to return the fluid to a substantially normal pH (“substantially normal,” as used herein, means not significantly different from normal, i.e., about 7.3 to 7.4). For example, a weak acid cab be administered, or a buffer capable of returning the pH to the desired level. Carbonic anhydrase inhibitors and bicarbonate transporter inhibitors can also be used, e.g., acetazolamide, celecoxib, valdecoxib, topiramate, and zonisamide; see, e.g., Morgan et al., Mol. Memb. Biol., 21:423-433 (2004).

pH-Based Diagnostics

In addition, the present inventors have shown that pH can be used as a diagnostic of the presence of, or increased risk of developing, a disorder associated with increased RVP as described herein.

Methods known in the art and described herein can be used to determine extracellular pH, e.g., in the vitreous or in the CSF. In some embodiments, the pH is measured in situ in a living mammal. For example, a microminiature pH-sensing electrode can be inserted via an opening through the sclera; the use of a glass electrode is described in Pedersen et al. Acta Ophthalm. Scand. 84(4):475 (2006). Numerous fluorescent dyes (see, e.g., Med. Biol. Eng. Comput. 32(2):224-7 (1994)), including 2′,7′-bis-(2-carboxyethyl)-5-(and-6)-carboxyfluorescein (BCECF), e.g., conjugated to dextran, can also be used, and a fluorescent image obtained using known in vivo imaging methods. Extracellular pH can also be determined in the brain of a living mammal, e.g., using MRI spectroscopy and pH-dependent relaxivity. See, e.g., Garcia-Martin et al., Mag. Res. Med. 55:309-315 (2006). ³¹P NMR spectroscopy, ion-sensitive field-effect transistors, and miniature biosensors are all known in the art and can be used to measure pH in vivo. See, e.g., Yuqing et al., J. Biochem. Biophys. Methods. 63(1):1-9 (2005); Marzouk et al., Anal. Biochem. 308(1):52-60 (2002).

In addition, pH can also be measured in a sample of fluid, e.g., a sample of vitreous or cerebral fluid, using methods known in the art, including pH-sensing microelectrodes and pH sensitive probes.

In general, a “normal” pH is about 7.4, e.g., is not statistically significantly different from 7.4. The determination of a threshold level for elevation can be performed using standard statistical methods. In some embodiments, the presence of a pH of about 7.8 or above is considered significantly elevated.

In some embodiments, the presence of an elevated pH in the eye or brain of a subject is indicative that the subject should be treated for, or treated to decrease risk of developing, a condition associated with increased RVP, e.g., using a method described herein.

### Formulation and Administration of Pharmaceutical Compositions

The methods described herein can include the administration of pharmaceutical compositions, which typically include the active ingredient and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is typically formulated to be compatible with its intended route of administration. Examples of routes of administration include parenteral, e.g., intravenous, intradermal, subcutaneous, oral, or nasal (e.g., inhalation), intrathecal (e.g., subdural or subarachnoid), transdermal (topical), transmucosal, and rectal administration. In some embodiments, e.g., for treating disorders associated with excessive retinal vascular permeability, the composition is administered directly to the eye, e.g., by eye drops, or directly into the eye across the blood-retinal barrier, e.g., by implants, peribulbar injection, or intravitreous injection. In some embodiments, e.g., for treating disorders associated with excessive cerebral vascular permeability, the composition is delivered across the blood-brain barrier, e.g., intrathecal, e.g., subdural or subarachnoid delivery, e.g., delivery into the cerebral or cerebrospinal fluid. In some embodiments, e.g., for administration to the vitreous or retina, the active ingredient is incorporated into a polymer matrix that is implanted into or near the site of intended delivery.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or basis, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol or sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the composition is especially adapted for administration into or around the eye. For example, a composition can be adapted to be used as eye drops, or injected into the eye, e.g., using peribulbar or intravitreal injection. Such compositions should be sterile and substantially endotoxin-free, and within an acceptable range of pH. Certain preservatives are thought not to be good for the eye, so that in some embodiments a non-preserved formulation is used. Formulation of eye medications is known in the art, see, e.g., Ocular Therapeutics and Drug Delivery: A Multi-Disciplinary Approach, Reddy, Ed. (CRC Press 1995); Kaur and Kanwar, Drug Dev Ind Pharm. 2002 May; 28(5):473-93; Clinical Ocular Pharmacology, Bartlett et al. (Butterworth-Heinemann; 4th edition (Mar. 15, 2001)); and Ophthalmic Drug Delivery Systems (Drugs and the Pharmaceutical Sciences: a Series of Textbooks and Monographs), Mitra (Marcel Dekker; 2nd Rev&Ex edition (Mar. 1, 2003)).

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Administration of a therapeutic compound described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Compositions including nucleic acid compounds can be administered by any method suitable for administration of nucleic acid agents. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996). In some embodiments, the nucleic acid compounds comprise naked DNA, and are administered directly, e.g., as described herein. The inhibitory nucleic acid molecules described herein can be administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription and/or translation. Alternatively, inhibitory nucleic acid molecules can be modified to target selected cells and then administered systemically. For systemic administration, inhibitory nucleic acid molecules can be modified such that they specifically bind to receptors or antigens expressed on a selected cell surface, e.g., by linking the inhibitory nucleic acid nucleic acid molecules to peptides or antibodies that bind to cell surface receptors or antigens. The inhibitory nucleic acid nucleic acid molecules can also be delivered to cells using the vectors described herein. To achieve sufficient intracellular concentrations of the inhibitory nucleic acid molecules, vector constructs in which the inhibitory nucleic acid nucleic acid molecule is placed under the control of a strong promoter can be used.

In some embodiments, the compositions are prepared with carriers that will protect the active ingredient against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially, e.g., from Alza Corporation or Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The delivery systems can include time-release, delayed release or sustained release delivery systems. Such systems can avoid repeated administrations of the agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109.

Delivery systems can also include non-polymer systems, e.g., lipids including sterols such as cholesterol, cholesterol esters and fatty acids or neutral fats such as mono-, di- and tri-glycerides; hydrogel release systems; sylastic systems; peptide based systems; wax coatings; compressed tablets using conventional binders and excipients; partially fused implants; and the like. Specific examples include, but are not limited to erosional systems in which the active agent is contained in a form within a matrix such as those described in U.S. Pat. Nos. 4,452,775, 4,667,014, 4,748,034 and 5,239,660, and diffusional systems in which an active component permeates at a controlled rate from a polymer such as described in U.S. Pat. Nos. 3,832,253, and 3,854,480. Pump-based hardware delivery systems can be used, some of which are adapted for implantation. In addition, U.S. Pat. No. 6,331,313 describes a biocompatible ocular drug delivery implant device that can be used to deliver active agents directly to the macular region.

Use of a long-term sustained release implant may be particularly suitable for treatment of chronic conditions. Long-term release means that the implant is constructed and arranged to delivery therapeutic levels of the active ingredient for at least 30 days, e.g., 60 days. Long-term sustained release implants are known to those in the art and include some of the release systems described herein.

The pharmaceutical compositions can be included in a container, pack, or dispenser together with instructions for administration.

### Methods of Treatment

The pharmaceutical compositions described herein are useful in the treatment of disorders associated with increased vascular permeability, as described herein.

As used in this context, to “treat” means to ameliorate at least one symptom of the disorder associated with increased vascular permeability. Often, increased systemic vascular permeability results in capillary leak syndrome and hypovolaemia; thus, a treatment can result in a reduction in capillary leakage and a return or approach to normovolemia. Administration of a therapeutically effective amount of a composition described herein for the treatment of a condition associated with increased vascular permeability will result in decreased vascular permeability. In diabetic retinopathy, administration of a therapeutically effective amount of a composition described herein may result in unobstructed vision, improved vision or reduction in the rate of visual loss.

Dosage, toxicity and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in a method described herein, the therapeutically effective dose can be estimated initially from animal studies, e.g., from intravitreal injection in animals. A dose may be formulated in animal models to achieve a circulating plasma concentration range that includes the IC50 (i.e., the concentration of the test compound which achieves a half-maximal inhibition of symptoms) as determined in intravitreal injection. Such information can be used to more accurately determine useful doses in humans. Levels in plasma or vitreous may be measured, for example, by high performance liquid chromatography and mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

### Methods of Screening

Also described herein are methods for screening test compounds, e.g., polypeptides, peptides, polynucleotides, inorganic or organic large or small molecule test compounds, to identify agents useful in the treatment or prevention of opthalmological disorders associated with increased retinal vascular permeability, e.g., diabetic retinopathy.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The small molecules can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the small organic molecules and libraries thereof can be obtained by systematically altering the structure of a first small molecule, e.g., a first small molecule that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a cell or living tissue or organ, e.g., an eye, and one or more effects of the test compound is evaluated. In a cultured or primary cell for example, the ability of the test compound to inhibit PK, FXII, or HK, or to modulate the pH of the cells to substantially normal, can be evaluated. In the eye, for example, the ability of the test compounds to inhibit PK, FXII, or HK, or to modulate the pH of the vitreous to substantially normal, can be evaluated.

To identify inhibitors of PK, FXII, or HK, the test sample can include all of the components of the contact system, e.g., PK, FXII, and HK, along with a chromogenic substrate, which allows the detection of amidolytic activity. In general, the assay will be carried out in a liquid sample, in the presence of purified polypeptides, the test sample, and a chromogenic substrate, e.g., as described herein, e.g., a fluorogenic kallikrein substrate such as H-D-Val-Leu-Arg-AFC, Calbiochem.

In some embodiments, the test sample is an “engineered” in vivo model. For example, vitreous from a human subject, e.g., a human subject having diabetic retinopathy, can be transplanted into one or both eyes of an animal model, e.g., a rodent such as a rat. For example, about 10 μl of human vitreous can be injected into the rat vitreous compartment and the response on retinal vascular permeability measured. Alternatively or in addition, purified PK, FXII, and HK can be injected. In some embodiments, the model animal also has diabetes, e.g., a streptozotocin-induced or genetic animal model of diabetes. In some experiments, the polypeptides or human vitreous will be co-injected with other agents, e.g., test compounds, such as known or potential inhibitors of PK, FXII, or HK.

Test compounds identified as “hits” (e.g., that inhibit the contact system) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating opthalmological disorders associated with increased retinal vascular permeability, as described herein, e.g., diabetic retinopathy. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of an opthalmological disorder associated with increased vascular permeability, as described herein. The animal can be monitored for a change in the disorder, e.g., for an improvement in a parameter of the disorder, e.g., a parameter related to clinical outcome. In some embodiments, the parameter is vascular permeability, and an improvement would be a decrease in vascular permeability. In some embodiments, the subject is a human, e.g., a human with diabetes, and the parameter is visual acuity.

### EXAMPLES

The invention is further described in the following examples, which do not limit the scope of the invention described in the claims.

### Example 1

### CA-1 Induced Increases in Retinal Vascular Permeability are Mediated by the Kallikrein-Kinin Pathway

The role of carbonic anhydrase 1 (CA1) and C1-inhibitor (C1-INH) in retinal vascular permeability (RVP) has been previously described, see International Patent Application No. PCT/US2006/0053, filed Feb. 16, 2006 and published as WO 2006/091459; the entire contents of that application are incorporated by reference herein. As shown therein, high levels of carbonic anhydrase-1 (CA-1) were identified in vitreous from patients with advanced diabetic retinopathy. Intravitreal injection of CA-1 in rats increased retinal vascular permeability. This response was comparable in magnitude, additive to vascular endothelial growth factor (VEGF)-induced permeability, and blocked by C1 inhibitor (C1-INH) and antagonists of the kallikrein-bradykinin receptor pathway. Further, carbonic anhydrase inhibition by acetazolamide blocked the increased retinal permeability in rats induced by transplant of vitreous from patients with advanced diabetic retinopathy. Therefore, carbonic anhydrase was shown to be a novel physiological activator of the contact/kallikrein system via a C1-INH-sensitive protease pathway, and plays a major role in the retinal vascular permeability in diabetic retinopathy.

In the present example, the role of the kallikrein-kinin pathway in CA-I induced RVP was examined further, in part to evaluate the possibility of targeting prekallikrein (PK) to modulate RVP. The vitreous of live rats' eyes were injected with balanced saline solution (BSS), 20 ng human erythrocyte CA-I (Sigma), with or without 5 μl of 0.66 mg/ml anti-prekallikrein (anti-PK) antibody (Abcam) in 10 μl final volume. Video fluorescein angiography was performed using a scanning laser opthalmoscope (Rodenstock Instrument). Vitreous fluorophotometry was performed as previously described (Aiello et al., Diabetes 1997; 46:1473-1480).

The results, shown in FIG. 1, demonstrate that co-injection of anti-PK antibody, which sterically blocks activation of PK by FXIIa (see Veloso et al., Blood 70:1053-1062 (1987)), blocked CA-I-stimulated RVP 81%. In contrast, anti-PK antibody pretreatment did not affect RVP stimulated by intravitreal injection of VEGF (FIG. 1).

The effects of C1-INH and anti-PK antibody on the kinetics of PK activation by factor XII (FXII) were monitored in vitro using a fluorescent kallikrein substrate. Fluorogenic kallikrein substrate (H-D-Val-Leu-Arg-AFC; Calbiochem) was used to quantify kallikrein enzymatic activity. Factor XIIa substrate (D-cyclohydrotyrosyl-glycyl-L-arginine-para-nitroanilide diacetate salt; American Diagnostica) was used to quantify FXIIa enzymatic activity produced following prekallikrein, kallikrein-mediated FXII activation, or FXII autoactivation.

Amidolytic activity of Factor XIIa was measured by 0.5 mM FXIIa substrate in the presence of 20 μM 2-Tosylamino-4-phenylbutyric acid-(4′-amidinoanilide) hydrochloride (American Diagnostica). Briefly, Factor XIIa substrate (D-cyclohydrotyrosyl-glycyl-L-arginine-para-nitroanilide diacetate salt; American Diagnostica) was used to quantify FXIIa enzymatic activity produced following prekallikrein, kallikrein-mediated FXII activation, or FXII autoactivation.

The effect of pH on FXII activation by PK or kallikrein was measured in 100 μl HEPES (10 mM) buffer containing 40 nM human FXII, 40 nM HK, 40 nM human PK or 20 nM human kallikrein and 5 mg/ml kaolin (Sigma). Reactions were incubated 3 min (for PK) or 10 min (for kallikrein) at room temperature, centrifuged to spin down kaolin, and the amidolytic activity of the supernatant was measured using 0.5 mM FXIIa substrate in the presence of 20 μM 2-tosylamino-4-phenylbutyric acid-(4′-amidinoanilide) hydrochloride (i.e., a synthetic inhibitor of plasma kallikrein; American Diagnostica).

The influence of pH on the relative amount of autoactivated 200 nM FXII generated during a 30 min incubation at room temperature in the presence of 5 μg/ml dextran sulfate (Sigma) was analyzed in 100 μl HEPES (10 mM) buffer. Amidolytic activity was measured by the absorbance change at 405 nm using a microplate reader (VICTOR3 V™ Multilabel Counter; PerkinElmer).

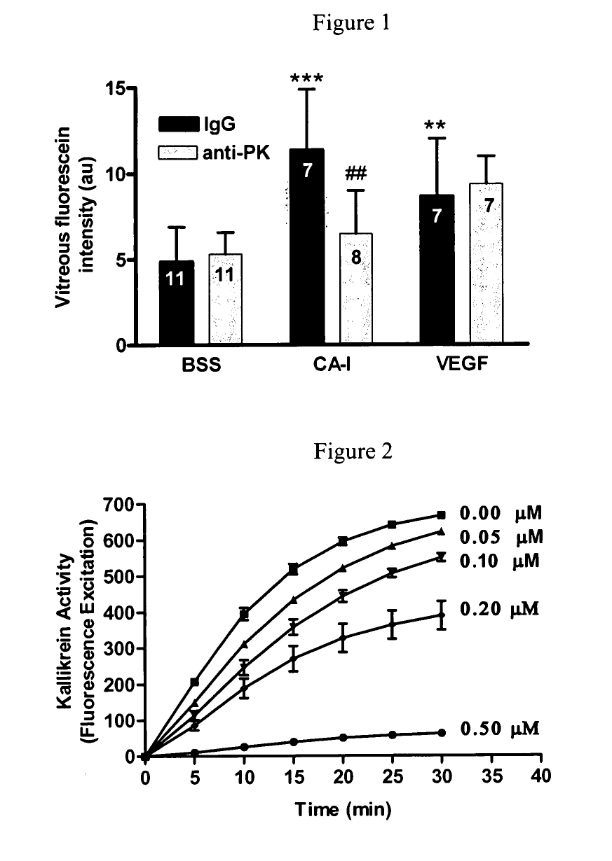

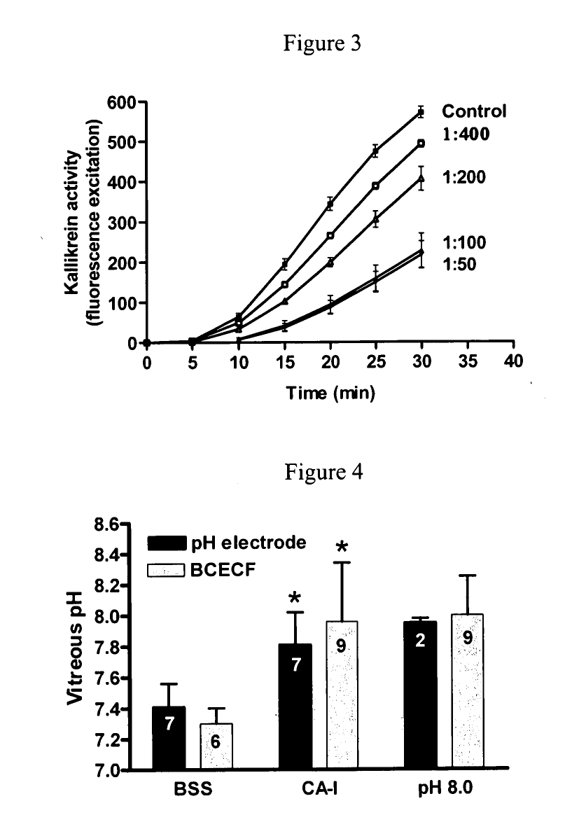

These studies confirmed that PK activation by CA-1 is inhibited by C1-INH and anti-PK antibody (see FIGS. 2 and 3), and that CA-1 induced increases in RVP can be reduced or eliminated by inhibitors of PK.

### Example 2

### Alkaline pH Mimics the Effects of CA-1

CA plays a central role in the regulation of extracellular pH and the retina contains robust mechanisms for ion transport. The effect of CA-I on vitreous pH was measured using a microminiature electrode via an opening through the sclera or a pH sensitive fluorescent probe, 2′,7′-bis-(2-carboxyethyl)-5-(and-6)-carboxyfluorescein (BCECF) conjugated to 70 kDa dextran. Using the electrode, we found that the pH of vitreous 5 min after injection of CA-1 was 7.8±0.21 compared with the pH of 7.4±0.15 following injection of BSS vehicle (P<0.002) (FIG. 4).

This electrode was also used to directly measure the pH of vitreous following an intravitreal injection with BCECF-dextran dye in either BSS at pH 7.4 or HEPES buffer at pH 8.0 following measurement of vitreous fluorescence. Using a pH sensitive dye and reference pH, the vitreous pH was calculated to be 7.96±0.38 following intravitreal injection of CA-I.

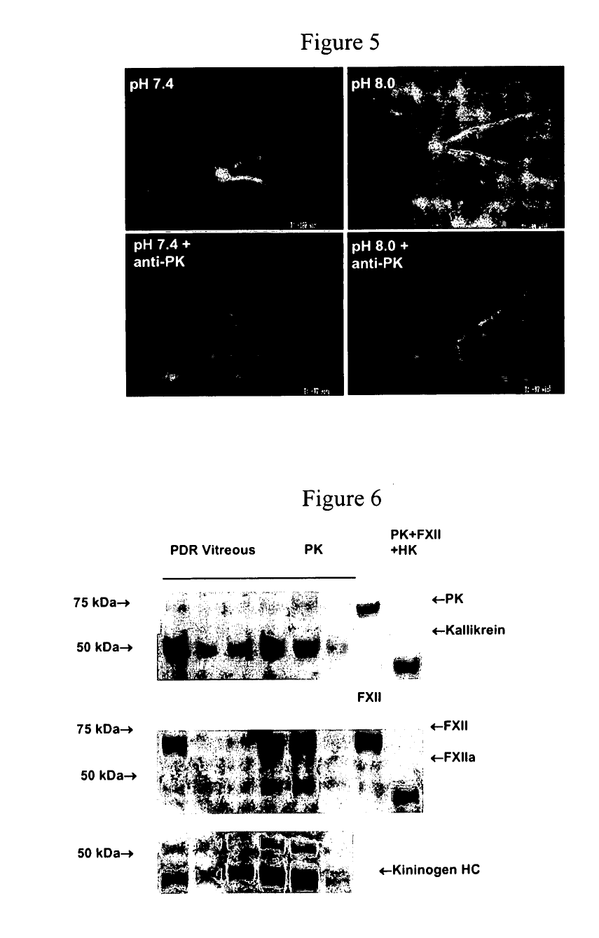

To determine the effect of an alkaline pH on RVP, BSS adjusted to pH 7.4 or 8.0 was injected into the vitreous and retinal fluorescein leakage was monitored by fluorescein angiography. Intravitreal injection of BSS at pH 7.4 did not produce a detectable effect on vascular leakage (FIG. 5). In contrast, intravitreal injection of BSS at pH 8.0 increased RVP to an extent comparable to that observed following CA-I injection. Intravitreal injection of the neutralizing anti-PK antibody reduced the increase in RVP induced by subsequent injection of BSS pH 8.0 (FIG. 5).

### Example 3

### The Contact System is Present and Activated in the Vitreous of Patients with PDR

Since the data described above showed that anti-PK antibody blocked both CA-I- and BSS pH 8.0-induced RVP (FIGS. 1 and 5), the presence of components of the kallikrein system in human vitreous was examined, and the effect of pH on the kallikrein pathway was investigated. Activation of the kallikrein system via the contact system involves both (i) kallikrein-mediated cleavage of FXII to FXIIa and (ii) FXIIa-mediated cleavage of PK to kallikrein. As shown in FIG. 6, vitreous from patients with PDR contains PK, kallikrein, FXII, FXIIa, and kininogen heavy chain.

High molecular weight kininogen (HK) is also present in the vitreous. Comparison of vitreous PK and FXII with 20 ng of purified PK or FXII controls indicates that PDR vitreous contains low μg/mL levels of these proteins. The appearance of both kallikrein and FXIIa in these samples suggests that the contact system is present and activated in the vitreous of patients with PDR.

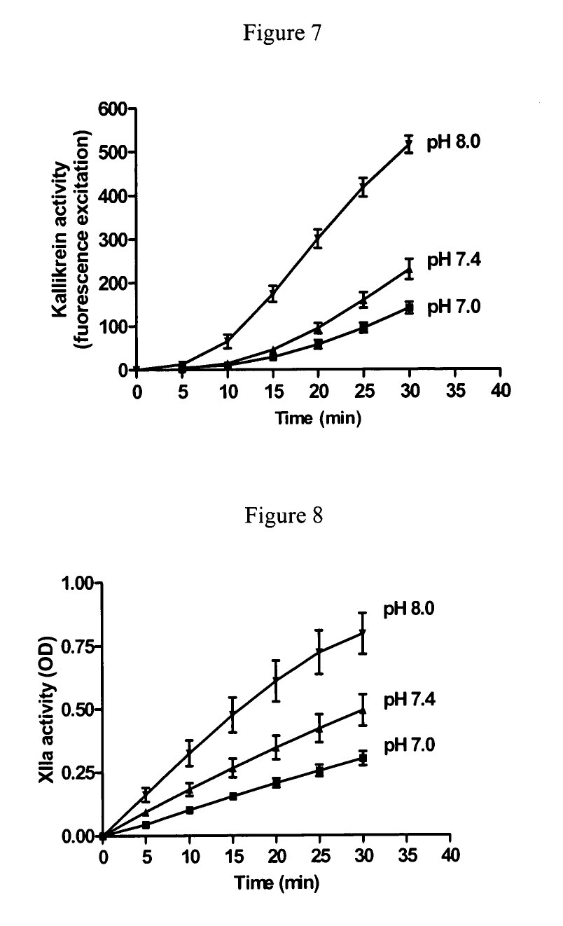

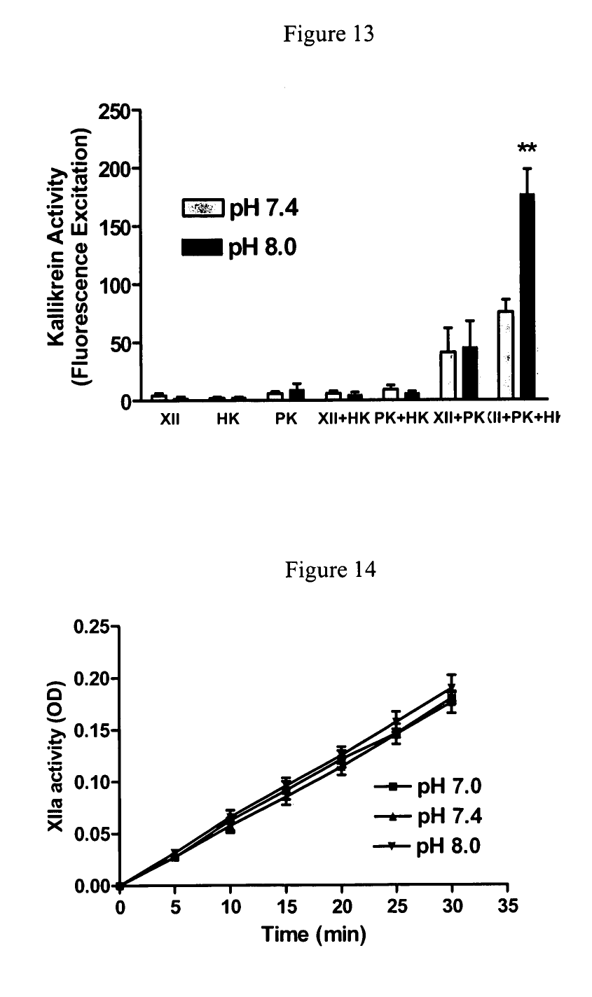

The effect of pH on the kinetics of PK activation in the presence of FXII was monitored in vitro using a fluorescent kallikrein substrate, as described above. The kinetics of PK activation in the presence of FXII and HK was facilitated by alkaline pH compared to neutral pH (FIG. 7). Neither CA-I or bicarbonate ion affected kallikrein activation in vitro (FIG. 12). Moreover, the increase in PK activation by alkaline pH required the combination of FXII, PK, and HK (FIG. 13). Alkaline pH increased FXII activation in the presence of PK and kaolin (FIG. 8), but did not affect FXII autoactivation in the absence of PK (FIG. 14).

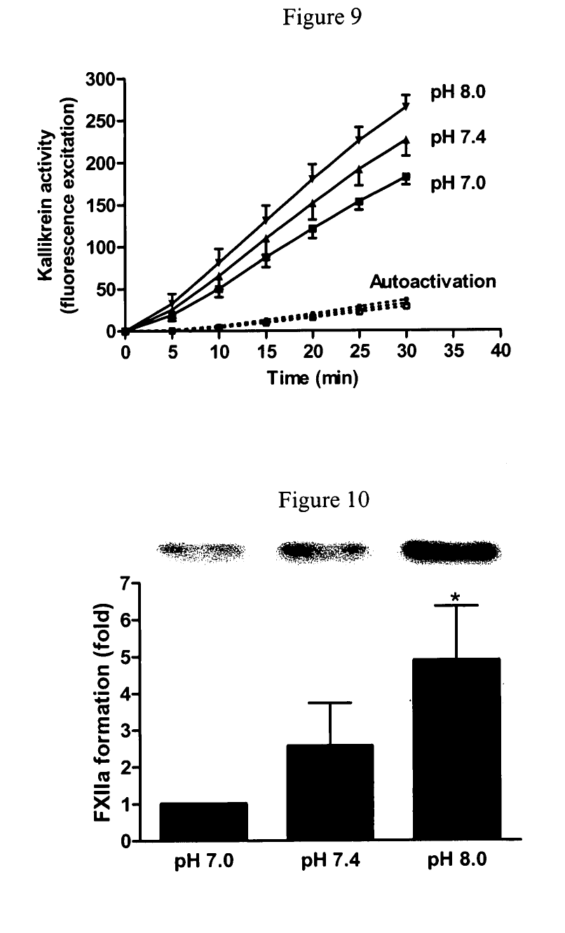

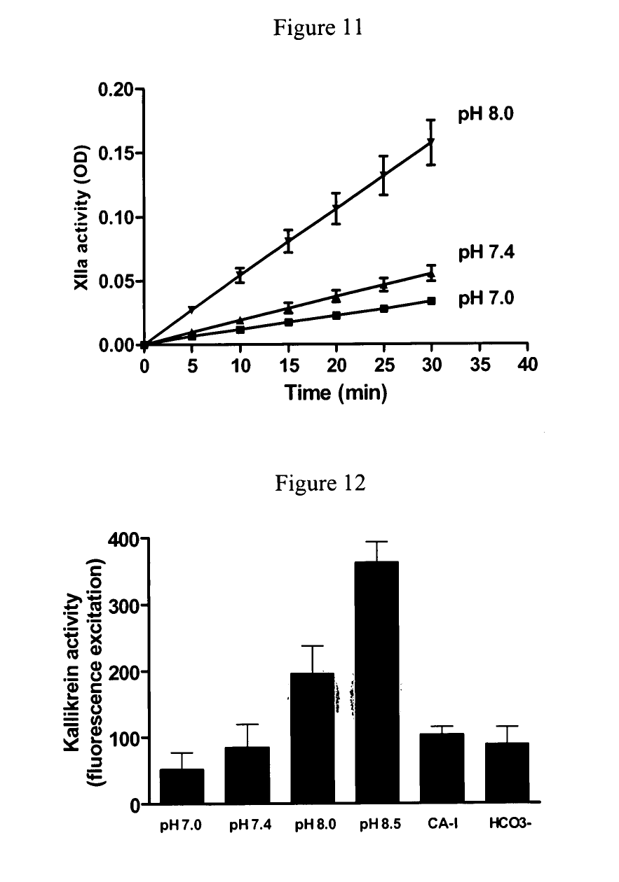

In contrast, kallikrein activity in the absence of FXII was increased at pH 8.0 compared with pH 7.4, but alkaline pH did not affect PK autoactivation (FIG. 9). Moreover, both kallikrein-mediated generation of FXIIa protein and FXIIa activity was enhanced by alkaline pH (FIGS. 10 and 11).

These results show that the pH sensitive event in kallikrein activation is the increase in kallikrein activity, which augments the generation of the PK activator FXIIa.

### Other Embodiments

All publications, patents, and patent applications, including U.S. Provisional Application No. 60/897,387, filed Jan. 25, 2007, mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating or preventing the development or progression of a condition associated with increased vascular permeability in the eye of a subject comprising:
a) optionally selecting a subject on the basis that they have a history of ocular hemorrhage or a condition associated with increased vascular permeability in the eye of a subject, and
b) administering to said subject a therapeutically effective amount of one or more of an inhibitor of Factor XII (FXII).

2. The method of claim 1, wherein said subject is a mammal.

3. The method of claim 1, wherein said subject is a human.

4. The method of claim 1, wherein said inhibitor is selected from the group consisting of a C1 inhibitor, a Corn Hageman Factor Inhibitor (CHFI), a H-D-Pro-Phe-Arg-chloromethylketone (PCK), a haemaphysilin, a hamandrin, an alpha 2-antiplasmin, an alpha 2-macroglobulin, an antithrombin III, an ecotin XII-18, and a plasma kallikrein-specific kunitz domain inhibitor (KALI-DY).

5. The method of claim 1, wherein said inhibitor is in combination with a pharmaceutically acceptable carrier.

6. A method of treating or preventing the development or progression of a condition associated with increased vascular permeability in the eye of a subject comprising:
a) optionally selecting a subject on the basis that they have a history of ocular hemorrhage or a condition associated with increased vascular permeability in the eye of a subject, and
b) administering to said subject a therapeutically effective amount of one or more of an inhibitor of prolylcarboxypeptidase, prekallikrein (PK), or high molecular weight kininogen (HK).

7. The method of claim 6, comprising administering one or more of HKH20, an inhibitory anti-PK antibody, an inhibitory anti-HK antibody, a benzamidine, a corn trypsin inhibitor, a diisopropyltluorophosphonate, a leupeptin, an anti-PRCP antibody, and a soybean trypsin inhibitor.

8. A method of treating or preventing the development or progression of a condition associated with increased vascular permeability in the eye of a subject comprising:
a) optionally selecting a subject on the basis that they have a history of ocular hemorrhage or a condition associated with increased vascular permeability in the eye of a subject, and
b) administering to said subject one or more of an antibody, or an antigen-binding portion thereof, with binding specificity for prekallikrein or heat shock protein 90, that inhibits binding between prekallikrein and heat shock protein 90.

9. The method of claim 8, wherein said antibody or antigen-binding portion thereof comprises a monoclonal antibody, a polyclonal antibody, a humanized antibody, a chimeric antibody, a single-chain Fv molecule, a bispecific single chain Fv ((scFv′)₂) molecule, a diabody, a triabody, a Fab fragment, a F(ab′)₂ molecule, or a tandem scFv (taFv) fragment.

10. A method for treating a subject who has a history of ocular hemorrhage comprising selecting a subject on the basis that they have had at least one ocular hemorrhage, and administering to said subject a treatment to reduce the risk or occurrence of future hemorrhages.

11. The method of claim 10, wherein said subject has an underlying medical condition selected from the group consisting of diabetes, sickle cell anemia, hypertension, and trauma.

12. The method of claim 10, wherein said treatment is selected from the group consisting of administration of one or more of an anti-hypertensive drug, administration of a composition comprising activated Factor VII, reduction or reversal of any anticoagulation medicaments used by the subject, and administration of isotonic fluids.

13. The method of claim 12, wherein said activated Factor VII comprises eptacog alfa.

14. A method of treating or preventing the development or progression of a condition associated with increased vascular permeability in the eye of a subject comprising:
a) optionally selecting a subject on the basis that they have a history of ocular hemorrhage or a condition associated with increased vascular permeability in the eye of a subject, and
b) administering to said subject a therapeutically effective amount of one or more of a compound that substantially normalizes pH in the eye of said subject.

15. The method of claim 14, wherein said compound is selected from the group consisting of a weak acid, a buffer capable of returning the pH to the desired level, a carbonic anhydrase inhibitor, and a bicarbonate transporter inhibitor.

16. The method of claim 14, wherein said bicarbonate transporter inhibitor is selected from the group consisting of acetazolamide, celecoxib, valdecoxib, topiramate, and zonisamide.

17. A method of determining whether a subject has or is at increased risk of developing a condition associated with increased retinal vascular permeability, the method comprising determining the pH in the eye of the subject, wherein the presence of a pH that is significantly higher than normal indicates that the subject has or is at risk of developing a condition associated with increased retinal vascular permeability.

18. The method of claim 17, wherein said determining the pH is performed in the vitreous of said eye.

19. The method of claim 17, wherein a pH above about 7.5 indicates that the subject has or is at risk of developing a condition associated with increased retinal vascular permeability.

20. The method of claim 17, wherein a pH of about 7.8 or higher indicates that the subject has or is at risk of developing a condition associated with increased retinal vascular permeability.
